(19) United States
(12) Patent Application Publication
(10) Pub. No.: US 2005/0101828 A1
(43) Pub. Date: May 12, 2005

(54) Magnetic therapy clothing articles and complex magnetic unit for use therewith

(76) Inventors: Billies B. Butler, Elgin, SC (US); Norma M. Higgins, Columbia, SC (US)

Correspondence Address: NELSON MULLINS RILEY & SCARBOROUGH, LLP, 1320 MAIN STREET, 17TH FLOOR, COLUMBIA, SC, 29201, US

(21) Appl. No.: 10/809,283

(22) Filed: Mar. 25, 2004

Related U.S. Application Data
(63) Continuation of application No. 10/135,876, filed on Apr. 30, 2002, now abandoned.
(60) Provisional application No. 60/287,646, filed on Apr. 30, 2001.

(51) Int. Cl. A61N 2/00

(52) U.S. Cl. 600/15

(57) ABSTRACT

An article of clothing adapted to be worn on a wearer's body includes at least one complex magnetic therapy unit (CMU). The CMU is carried by the article of clothing so as to be positioned at a predetermined location on the wearer's body and thereby provide therapeutic effects.

This application claims priority to provisional application Ser. No. 60/287,646, filed Apr. 30, 2001, incorporated herein by reference.

### BACKGROUND OF THE INVENTION

The present invention relates generally to articles of clothing incorporating magnet units for use in magnetic therapy.

The continued use of magnetic therapy to provide relief of pain and to increase the flow of blood has been well documented. Although this proven practice has been successfully utilized throughout many countries around the world, it has only been in recent years that magnetic therapy has rapidly been gaining support in the United States.

Because of the effectiveness of magnetic therapy, many permanent magnetic products for providing magnetic therapy have been developed. Sports companies such as Nike and MacGregor Sports, as well as health care clinics nationwide, are selling magnetic health products. A good example of this is the golf-industry, which has become enamored of magnetic bracelets and wraps, touting their abilities to improve a golf game. In addition, there is an array of magnetic therapy products available to the consumer. These include golf gloves, sleep pads, wraps for various parts of the body (such as the knee, ankle, back, calf and wrist), headache and stress patches, nerve patches, eye strain clothes, magnetic jewelry, equine treatments for bruises, etc.

Various patents have also been granted showing magnetic therapy devices. For example, U.S. Pat. No. 5,720,046, incorporated herein by reference, discloses various articles of clothing that include permanent magnets for therapeutic purposes. According to this patent, magnets are positioned in accordance with the ancient art of applied massage therapy known as Shiatsu. Shiatsu concentrates on specific areas of the body known to provide relief from pain, which are used by acupuncture and acupressure practitioners.

Other patents show the use of permanent magnets used in conjunction with other devices. For example, U.S. Pat. No. 4,509,219 teaches a sleeping mattress structure provided with permanent magnets. Similarly, U.S. Pat. No. 4,921,560 teaches a method of affixing permanent magnets to bed covering. Each of these patents is also incorporated here in by reference.

### SUMMARY OF THE INVENTION

The present invention recognizes and addresses various disadvantages of prior art constructions and methods.

In accordance with the present invention, a portable Complex Magnetic Therapy Unit (“CMU”) is provided for use in specially designed articles of clothing and therapeutic wraps. Articles of clothing utilizing the CMU include sewn pockets necessary to contain the CMUs. The pockets are positioned at specific locations as called for in accordance with the present invention so as to provide therapeutic energetic pulsed signals to the cells that need to be revitalized.

As a precisely developed magnetic therapy unit, the CMU may be used in therapeutic guidance for redesigned flow of magnetized particles throughout the body. Various articles of clothing utilizing the CMU are contemplated for wear by both humans and animals, such as dogs. Such articles include men's briefs, gloves, neck supports, sock-like garments and exercise suits. Each article incorporating one or more CMUs includes a precise placement structure and systematic design for optimal effect of each magnetic unit.

Advantageously, the CMU is a unique; non-invasive, non-chemical device that can be used in the treatment and/or prevention of various conditions. For example, specific embodiments of the present invention may be used in the treatment of erectile dysfunction, magnetic field deficiency syndrome, carpal tunnel syndrome, arthritis and sports injuries, along with the loss of magnetism in cells within the body. The CMU is be scientifically placed in articles of clothing worn on the areas of the body corresponding with a predetermined relief system to revitalize and separate the mass of blood cells that clump together during a loss of magnetic influence in the ions and the protons within the body.

In one embodiment of the present invention, the CMU is incorporated into a unique and effective glove. Utilizing Shiatsu meridian pressure points and acupuncture pressure points, the CMU is placed in precisely the locations of the hand and wrist to best distribute the ionized particles in the cells in this area of the body to effectively contain the deterioration of the cells traumatized in the affected area.

Objects, features and aspects of the present invention are provided by various combinations and subcombinations of the disclosed elements, as well as methods of utilizing same, which are discussed in greater detail below.

### BRIEF DESCRIPTION OF THE DRAWINGS

A full and enabling disclosure of the present invention, including the best mode thereof, to one skilled in the art is set forth more particularly in the remainder of the specification, including reference to the accompanying drawings, in which:

FIG. 1 illustrates the manner in which blood cells clump together when magnetism in the body cells is low, compared to how the cells circulate freely on their own when exposed to a CMU constructed in accordance with the present invention.

FIG. 2 diagrammatically illustrates the manner in which lines of standard magnetic energy flow from the North Pole to the South Pole on a continual basis.

FIG. 3 illustrates the manner in which lines of magnetism flow as it is conducted by the CMU. The construction of the CMU is such that it allows and encourages the re-entry of magnetic energy into the part of the body exposed to the Unit. Re-entry in and around the affected part of the body requiring additional magnetic energy for healing is critical for cells to function at their optimum level. Re-energized cells can repair themselves and stabilize the healing process.

FIG. 4A is a diagrammatic plan view of a preferred base unit of the CMU showing the wave magnetization pattern.

FIG. 4B is a diagrammatic plan view of the base unit of FIG. 4A with insert units.

FIG. 4C is a diagrammatic plan view of insert units 3 and 4.

FIG. 4D is a diagrammatic plan view of insert unit 2 with insert unit 4 located therein.

FIG. 5 is front view of a male brief constructed in accordance with one embodiment of the present invention showing the placement of multiple CMUs. In this case, the briefs are standard length low-rise briefs and CMUs are placed at the following three locations: under the waistband, and on the left and right sides of the briefs.

FIG. 6 is a back view of the brief of FIG. 5. As can be seen, a pair of CMUs is placed below the waistband in two separate pockets.

FIG. 7 shows the palm side of a user's hand in open position. Placement of magnets is shown as they will come in contact with the hand and wrist when a glove constructed in accordance with a preferred embodiment of the present invention is worn.

FIG. 8 shows the same view of the open hand as in FIG. 7 with glove in place. In this embodiment, the finger portion of the glove extends over the knuckle of the finger and ends between the first and second joints on each finger. The glove extends back over the palm hand and over the wrist, extending 40 mm beyond the bend of the wrist.

FIG. 9 illustrates a neck support garment containing CMUs in accordance with the present invention being worn about the neck of a user. In presently preferred embodiments, the neck support garment may be approximately five inches from top to bottom edge. The garment closes in the back using a suitable attachment mechanism, such as hook and pile fasteners (i.e., Velcro).

FIG. 10 illustrates the neck support of FIG. 9 removed from the wearer's neck and opened to show a preferred placement for the multiple CMUs.

FIG. 11 illustrates the neck support of FIG. 9 removed from the wearer's neck and closed with Velcro fastened in the back to show the placement of the CMUs when the garment is worn.

FIG. 12 illustrates a sock-like garment containing CMUs in accordance with the present invention being worn on the foot and leg of a user.

FIG. 13 shows the leg of the user wearing the sock-like garment of FIG. 12 illustrating a preferred placement of CMUs.

FIG. 14 shows a preferred placement of CMUs in the sole of the sock-like garment of FIG. 12.

FIG. 15 illustrates an exercise suit containing CMUs in accordance with the present invention being worn on the body of a user.

FIG. 16 is a view of the exercise suit of FIG. 15 showing elastic straps sewn into the inside of the suit (the straps would not be seen from the outside of the garment). The internal straps are separated into an upper unit 1 and a lower unit 2 which are fastened together with a suitable attachment mechanism such as hook and pile fastener (i.e., Velcro) to form a complete unit. The white spots on the straps indicate a preferred placement location for the individual CMUs.

FIGS. 17A and 17B are enlarged front views of upper unit 1 and lower unit 2 of the exercise garment, respectively. The units are interconnected at Velcro attachment flaps FC1 and FC2.

FIGS. 18A and 18B are enlarged back views of upper unit 1 and lower unit 2 of the exercise garment, respectively. The units are interconnected at Velcro attachment flaps BC1 and BC2.

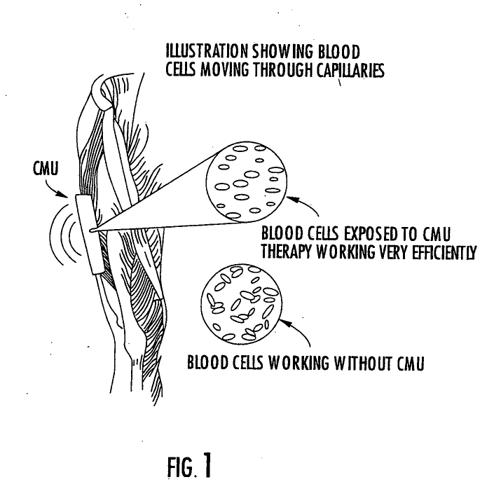

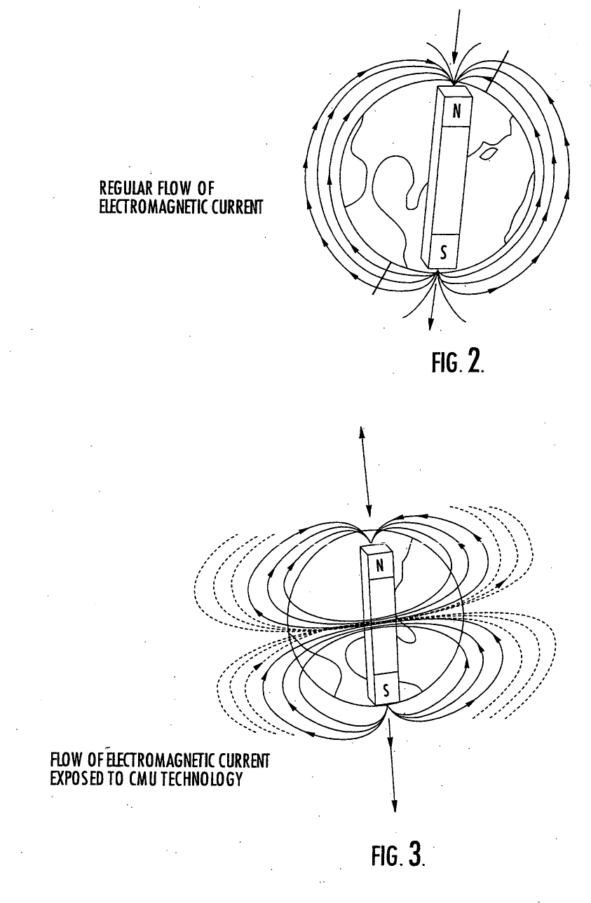

### DETAILED DESCRIPTION OF PREFERRED EMBODIMENTS

It is to be understood by one of ordinary skill in the art that the present discussion is a description of exemplary embodiments only, and is not intended as limiting the broader aspects of the present invention, which broader aspects are embodied in the exemplary constructions.

A. Overview of CMU Therapy

It should be made clear that magnets themselves do not heal anything—they only stimulate the body to heal itself. Magnetism is a wholly natural event. It is neither magic nor medicine. It merely allows body cells to exist at their optimum level.

In the treatment of sprains, strains, broken bones, burns and cuts, not only does magnetic field therapy aid in the recovery, but it allows these conditions to heal better, more quickly, and with less scar tissue. Magnetic treatment has been shown to decrease healing time by half or more.

In the treatment of chronic conditions such as some forms of arthritis, degenerative joint conditions, diabetic ulcers and cancer, magnetic field therapy has shown dramatic results in aiding the reduction or reversal of the condition.

Another effect of a magnetic field that is well documented and supported by the laws of physics is the enhancement of blood circulation and lymphatic drainage. The circulatory effect is documented in Faraday's Law and the Hall Effect, two long-accepted laws of physics that explain the principles by which the action of the magnetic polarities create ionic currents and patterns, which in turn increase the diameter of the blood vessels and ease the movement of the blood through those vessels.

According to Dr. Buryl Payne, a physicist, psychologist, and inventor of the first biofeedback instruments, sensitive research instruments have allowed scientists to document some of the ways magnetic fields affect living organisms. Among them are:

(1) Increased blood flow with resultant increased oxygen-carrying capacity, both of which are basic to helping the body heal itself.

(2) Magnetic fields change migration of calcium ions which can either bring calcium to heal a broken bone, or can help move calcium away from painful arthritic joints.

(3) The pH (acid/alkaline) of various body fluids (often out of balance in conjunction with illness or abnormal conditions) can apparently be altered by magnetic fields.

(4) Hormone production from the endocrine glands can be either increased or decreased to normal levels by magnetic stimulation.

As typical examples of specific effects created when a magnetic field is applied to the body, the following changes have been documented:
(a) Electricity is generated in blood vessels;
(b) Ionized particles increase in the blood;
(c) Autonomic nerves are excited; and
(d) Circulation is improved.

B. Specification for Preferred CMU

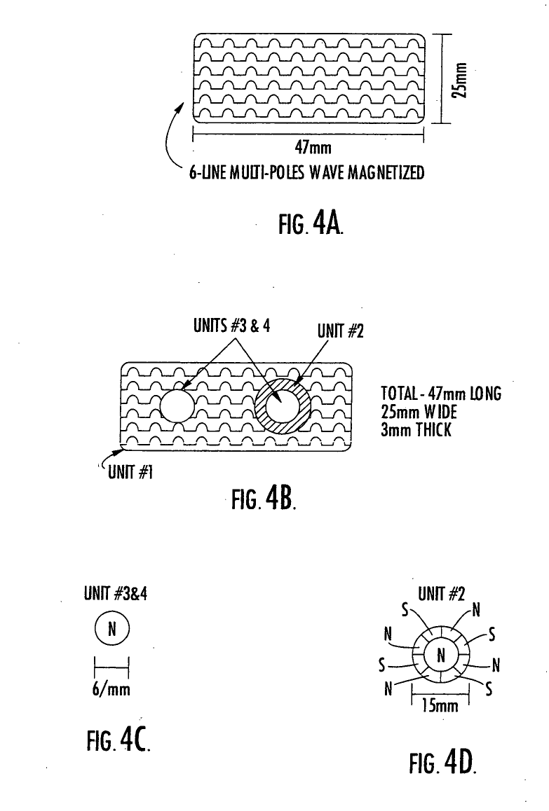

FIGS. 4A, 4B, 4C and 4D illustrate a preferred embodiment of a CMU constructed in accordance with the present invention. This exemplary CMU pattern has the following specifications:

Magnet #1 Base Unit
47 mm long with rounded corners;
25 mm wide;
3 mm thick;
850 gauss rating.

Magnet #2
16 mm round;
Placed in base magnet 5 mm from outside edge;
Open center;
6 alternating magnetized patterns (N and S poles) around surface of magnet;
600 gauss rating.

Magnet #3
6 mm round magnet placed in the center opening of magnet #2;
Will be North Pole facing up;
600 gauss rating.

Magnet #4
6 mm round magnet placed in the base magnet 5 mm from outside edge;
Will be North Pole facing up;
600 gauss rating.

C. Male Brief for Erectile Dysfunction

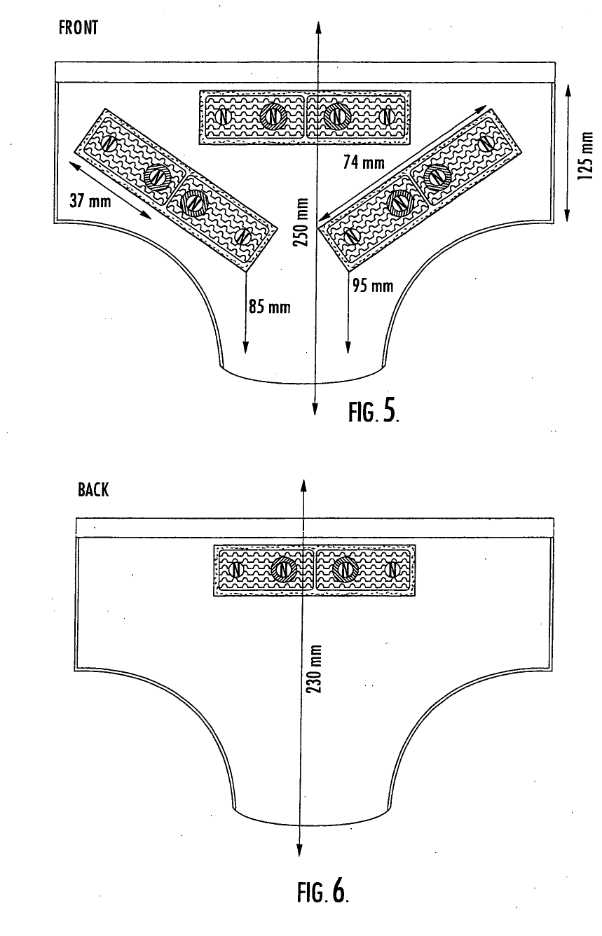

This article of clothing will be worn, on the lower torso of the male as underwear. The brief will replace the individual's normal underwear. Illustration of the briefs with measurements can be found in FIGS. 5 and 6.

Specifically, FIGS. 5 and 6 show exact placement of the CMUs into a preferred embodiment of the men's brief. As shown, a double pocket is preferably sewn into the brief 0.5 inch below the waistband. A CMU is placed into each of the cloth pockets which is then sewn closed on all four sides and down the middle section, separating the two CMUs. This keeps the CMUs in place.

FIG. 5 also shows that two additional double cloth pockets are preferably sewn into the brief, one double pocket on each side of the brief. These double pockets look and act as two single pockets just as the ones below the waistband. The CMUs are sewn next to each other with stitching around the outside edges and down the middle, separating the two CMUs. The bottom edge of the sewn pockets is pointing down toward the crotch area of the brief, as shown.

In accordance with one preferred construction, each of two pockets containing the CMUs on the left side of the brief extends 37 mm per pocket, making the length of the two pockets containing the CMUs a total of 74 mm. The bottom outer corner of the lower of the two pockets on the left side may preferably be 85 mm from the seam in the crotch area.

In the illustrated embodiment, the double pockets on the right side of the briefs containing the CMUs extend 37 mm per pocket, making the length of the two pockets containing the CMUs a total of 74 mm. The bottom outer corner of the lower of the two pockets on the right side may preferably be 95 mm from the seam in the crotch area.

As shown, each pocket contains one complete CMU in this case.

In addition, the length of the brief on both the left and right outside leg portion may preferably be exactly 125 mm from the top of the waistband to the bottom of the sewn seam at the leg opening.

In addition, the length of the brief from the top of the waistband down the middle of the brief to the center seam in the crotch area may preferably be exactly 250 mm on the front side of the briefs and 230 mm on the back side.

D. Glove Including CMUs

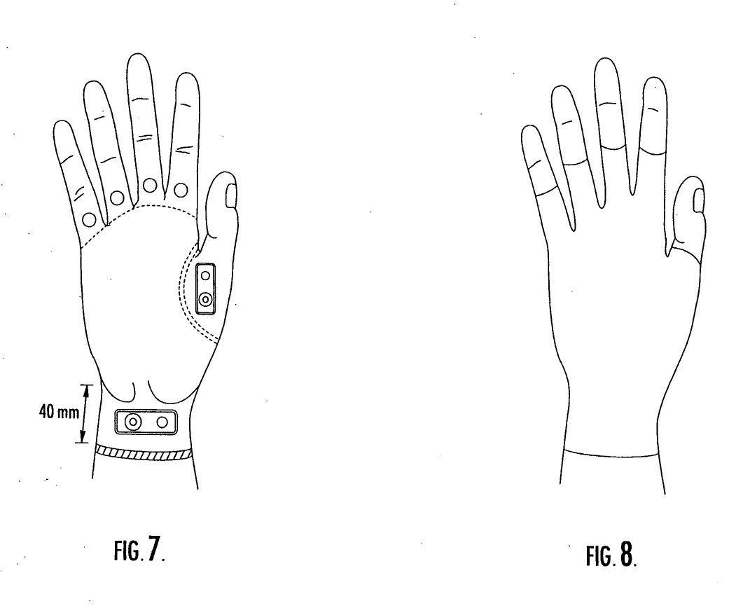

FIGS. 7 and 8 illustrate one preferred embodiment of a glove constructed in accordance with the present invention. The single dotted lines represent the location of the hand where fingers join the palm. The double dotted line represents the wrinkle that forms in the hand when the thumb is bent inward toward the fingers.

In this case, a CMU should preferably be placed just inside the wrinkle line on the thumb, 35 mm from the seam line. Round magnets on the fingers are placed just above the bend line on the hand between the fingers and the palm. In accordance with a preferred embodiment, the units are 10 mm round and 800 gauss each.

The CMU on the wrist may preferably be placed 10 mm behind the bend of the wrist where it connects with the palm. Moreover, the section of the glove extending over the wrist area should preferably be 40 mm long.

As shown, the glove extends from the base of the palm over the knuckle to halfway down the finger, ending between the first and second joint. The fingertips are cut out of the glove, providing greater use of the hand.

The glove may be used for positive pain relief for arthritis, carpal tunnel syndrome and sports injuries, as well as repetitive motion syndrome and other discomforts of the hand and wrist.

E. Neck Support Containing CMUs

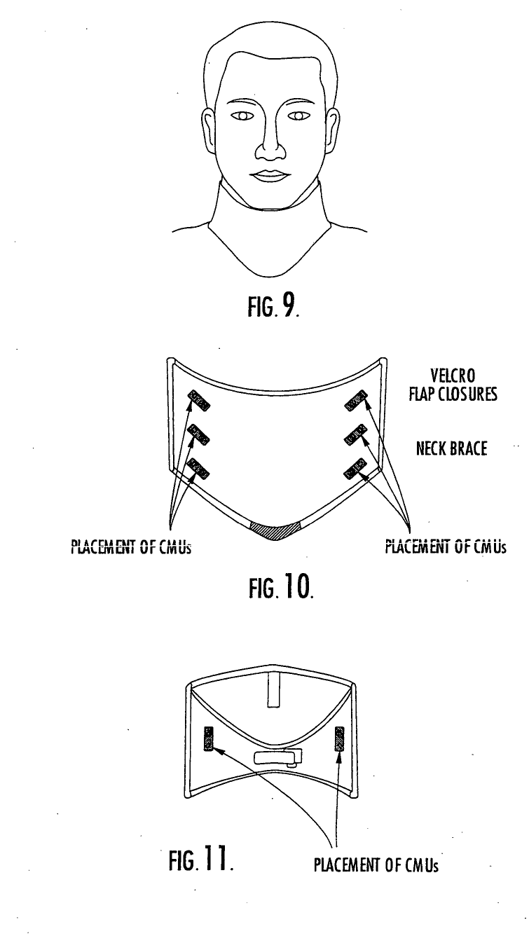

FIGS. 9 through 11 illustrate a preferred embodiment of a neck support garment including CMUs in accordance with the present invention. The illustrated neck support is designed to give substantial support to neck sprains or pain. In a preferred embodiment, the neck support is approximately six inches from top to bottom in the front and decreasing to about 2¼″ in the back where the Velcro will fasten. In this case, three CMUs are placed evenly on both the left and the right sides in the front of the neck support. One CMU may preferably be placed on both the left and right sides in the back placed evenly between the top and bottom. In a preferred embodiment, the neck support may be constructed of neoprene, open in the back with a Velcro closure which is 2¼″ wide.

F. Sock-Like Garment Containing CMUs

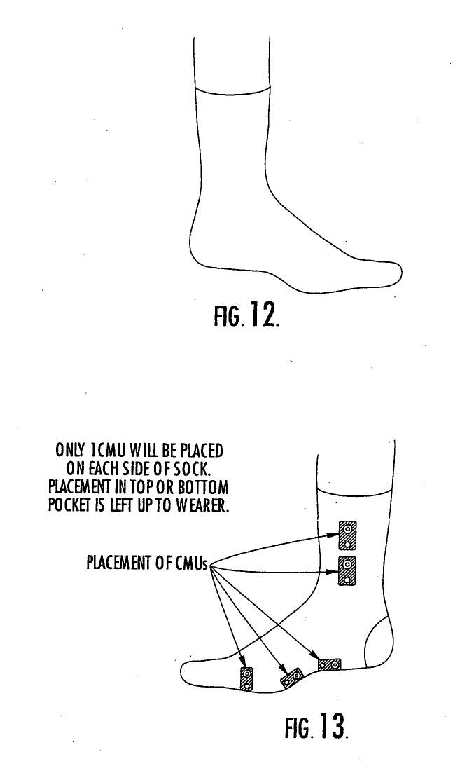

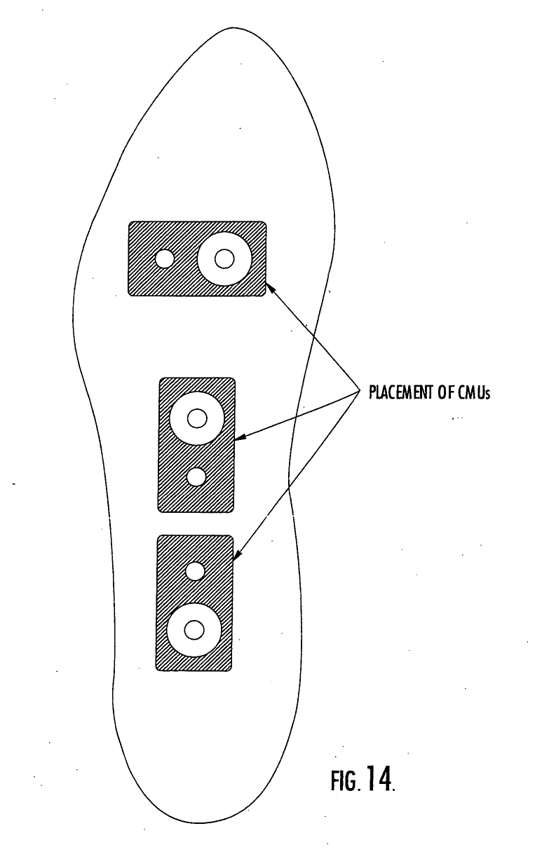

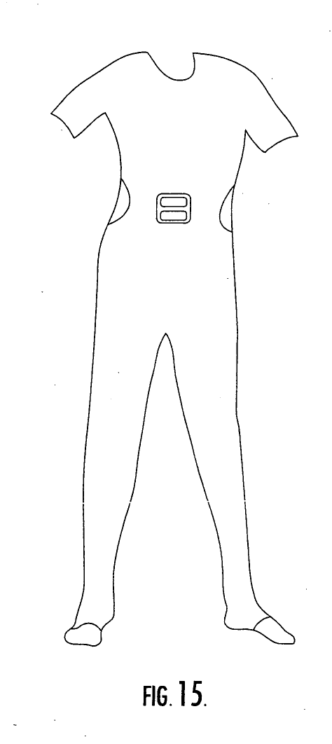

Referring now to FIGS. 12 through 14, a sock-like garment made according to a preferred embodiment of the present invention is illustrated. In this case, the garment may preferably be approximately 22″ long in tube form with no visible “foot or heel” section. On the left side of the sock, two (2) pockets may be sewn in, one above the other, for receipt of one CMU. (The user will place the single CMU in the pocket of choice.) Two (2) pockets are also sewn in, one above the other, on the right side of the sock for receipt of a single CMU in the user's pocket of choice.

Preferably, each set of pockets in the sock-like garment will be directly above the ankle, with the first pocket being 30 mm above the ankle area. In such embodiments, the bottom of the second pocket may begin 20 mm above the top of the first pocket. Preferably, each of the pockets will be approximately 53 mm by 30 mm.

The inner sole insert for the foot section of the sock-like garment will preferably be provided larger than needed to each purchaser so that it can be trimmed to fit the feet of that person. Preferably, the inner sole will be magnetized at a gauss rating of 800. In this regard, three (3) CMUs will preferably be placed in the following locations: (1) a first CMU may be placed 12 mm from the outside edge of innersole on the ball of the foot; (2) a second CMU will be placed in the center of the inner sole 35 mm from the point at which the edge of the first unit stops; and (3) a third CMU will be placed in the center of the inner sole 10 mm behind the second unit and 24 mm from the outside back edge of the inner sole.

Preferably, the overall sock-like garment will-be made from a suitable cotton blend material.

G. Exercise Suit Containing CMUs

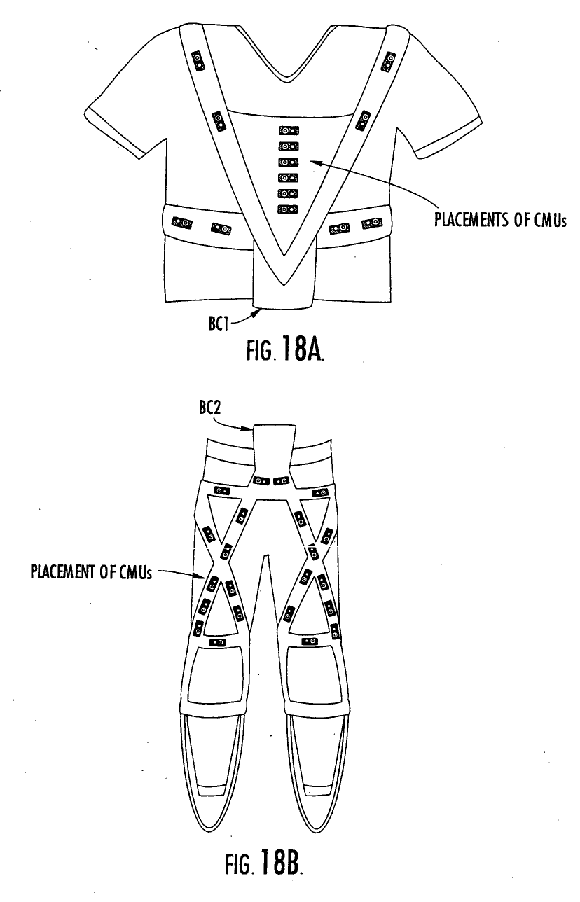

FIGS. 15 through 18B illustrate a preferred form of an exercise suit incorporating CMUs in accordance with the present invention. In this case, the exercise suit is constructed as a two-piece suit having a top unit and bottom unit that connect in the middle in both the front and back via Velcro closures, or other suitable attachment means.

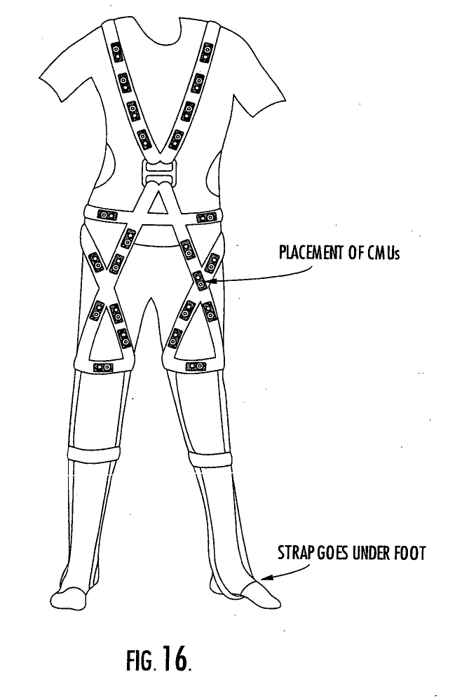

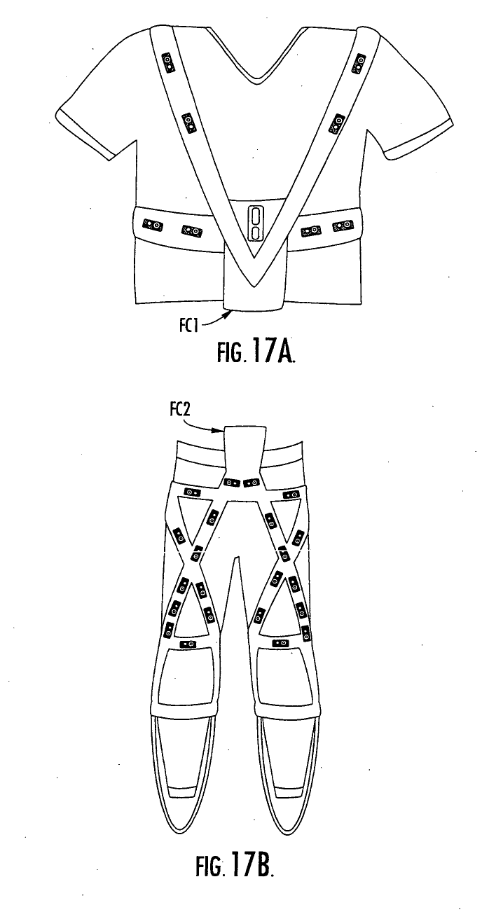

In a preferred embodiment, the top piece of the suit has 3″ wide straps running over the shoulders from front to back and meeting in the middle in the front and in the back. The straps each contain CMUs that are preferably placed according to the diagrams in FIGS. 16, 17A-B and 18A-B. As shown in FIG. 16, the straps in the bottom (pant) unit of the exercise suit will continue down the sides of the legs and cross over in the front.

While preferred embodiments of the invention have been shown and described, modifications and variations may be made thereto by those of ordinary skill in the art without departing from the spirit and scope of the present invention. In addition, it should be understood that aspects of the various embodiments may be interchanged both in whole or in part. Furthermore, those of ordinary skill in the art will appreciate that the foregoing description is by way of example only, and is not intended to be limitative of the invention so further described in such appended claims.

1. A combination comprising:
an article of clothing adapted to be worn on a wearer's body; and
at least one complex magnetic therapy unit carried by said article of clothing so as to be positioned at a predetermined location on said wearer's body and thereby provide therapeutic effects.
